(19) United States
(12) Patent Application Publication
(10) Pub. No.: US 2015/0134032 A1
(43) Pub. Date: May 14, 2015

(54) Method and Device for Application of Photobiomodulation Treatment

(71) Applicants: Mary T. Chicchi, Easton, PA (US); Adele M. Lukachek, Easton, PA (US)

(72) Inventors: Mary T. Chicchi, Easton, PA (US); Adele M. Lukachek, Easton, PA (US)

(21) Appl. No.: 14/080,396

(22) Filed: Nov. 14, 2013

(51) Int. Cl. A61N 5/06

(52) U.S. Cl. CPC A61N 5/0622; A61N 2005/0636; A61N 2005/067
USPC 607/89

(57) ABSTRACT

A device and method for application of photobiomodulation (or as low level laser therapy) treatment is disclosed. The device includes a canopy having a plurality of emitters configured and arranged to emit light in wavelengths between about 600 nm and about 1200 nm. A stand supports the canopy. The canopy configured and arranged pivot and raise and lower relative to the stand. During treatment, the entire first side of a patient is exposed to emitted light from the device therapeutically effective duration in a first step and the entire second side of a patient to emitted light for a therapeutically effective duration in a second step.

### BACKGROUND OF THE INVENTION

1. Field of the Invention

The present patent document relates generally to photobiomodulation, also known as low level laser therapy (LLLT) and devices and methods for applying LLLT treatment to a patient

2. Background of the Related Art

LLLT is an emerging technology, which is similar to the process of photosynthesis in plants. The technology involves using certain wavelengths of light to produce the following effects: (1) A Primary effect of absorption by chromophores, such as cytochrome c oxidase, which leads to (2) Secondary effect of displacement of nitric oxide, which reduces oxidative stress and increases ATP production, ultimately providing tertiary effects of (3) Increased metabolism, mitosis, reduced inflammatory markers, increased exchange of Ca2+, secretion of growth factors, activation of enzymes and other secondary messengers, and potential enhancement of neurorecovery mechanisms. Local cell-specific responses lead to systemic responses. In addition, there is also an analgesic effect when the mode is switched to inhibition rather than stimulation. The effects are at the dorsal root ganglia, dorsal horn, and the sympathetic ganglia of the nervous system.

Prior art treatments using LLLT devices are site specific. That is, they are designed to treat an arm or other part or portion of the patient. Therefore, there is a need in the art for a device and treatment protocol for applying LLLT to more than specific targeted treatment sites on a patient.

### SUMMARY OF THE INVENTION

The invention solves the problems of the prior art by providing a device and treatment protocol for applying LLLT to the entire body of a patient. The device includes a canopy having a plurality of emitters configured and arranged to emit light in wavelengths between about 600 nm and about 1200 nm. A stand supports the canopy. The canopy configured and arranged pivot and raise and lower relative to the stand.

During treatment, the entire first side of a patient is exposed to emitted light from the device for a therapeutically effective duration, in a first step, and the entire second side of a patient to emitted light for a therapeutically effective duration, in a second step. The patient may be treated while lying down or standing up. A patient is usually treated while lying down, however if a patient is unable to lie down due to an injury or paralysis, the device may be rotated vertically in order to treat a patient that is unable to lie down. Typically the durations of exposure last from about ten to thirty minutes.

### BRIEF DESCRIPTION OF THE DRAWINGS

These and other features, aspects, and advantages of the present invention will become better understood with reference to the following description, appended claims, and accompanying drawings where:

FIG. 1 is a front perspective view of a device for applying a LLLT treatment with the canopy in a horizontal orientation;

FIG. 2 is a rear perspective view of a device for applying a LLLT treatment with the canopy in a horizontal orientation;

FIG. 3 is a front perspective view of a device for applying a LLLT treatment with the canopy in a vertical orientation

FIG. 4 is a bottom view of the canopy; and

FIG. 5 is a flow diagram of a method of treatment using LLLT.

### DESCRIPTION OF THE PREFERRED EMBODIMENT

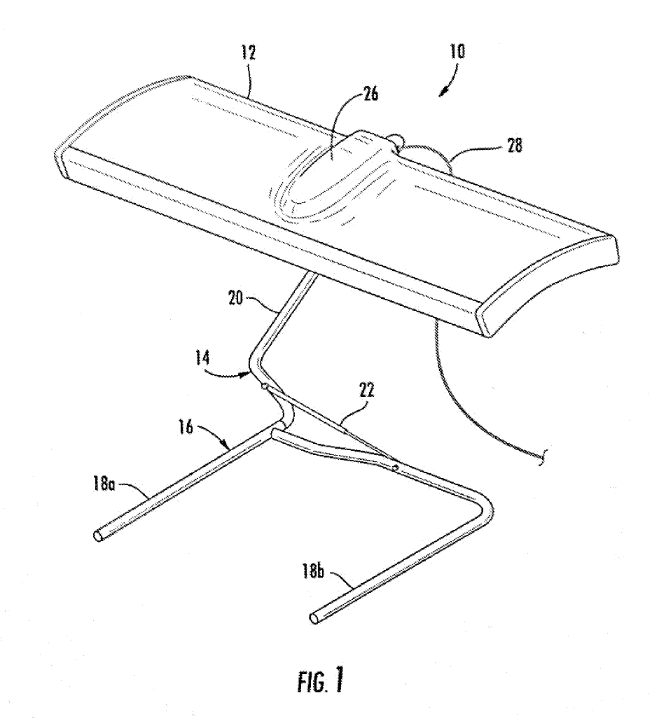

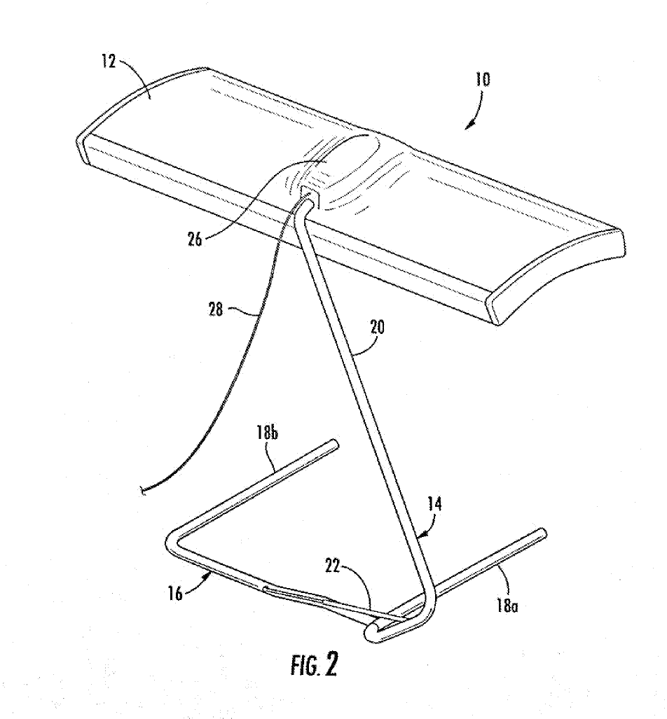

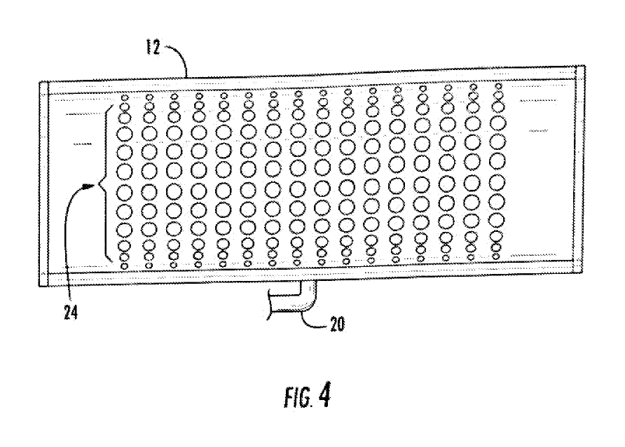

Referring now to FIGS. 1-4, the device for applying an LLLT treatment is shown generally at 10. The device 10 includes a canopy 12 pivotably supported on a stand 14, which is further configured to raise and lower the canopy 12 relative to the stand 14

The stand 14 has a base 16 with a pair of legs 18a, 18b. An arm 20 is received within on the legs 18a and is pivotable therewithin. A hydraulic cylinder 22 attached to the arm 20 and base 16 prevents the arm 20 from moving after it has been positioned by an operator. The canopy 12 is pivotably connected to the other end of the arm 20 permitting the canopy 12 to be oriented by an operator vertically, horizontally or at another desired angle as the case may be. The canopy 12 is large enough to expose the entire surface of a patient being treated. That is, roughly about 10,000 cm² to about 30,000 cm² depending upon the size of the patient.

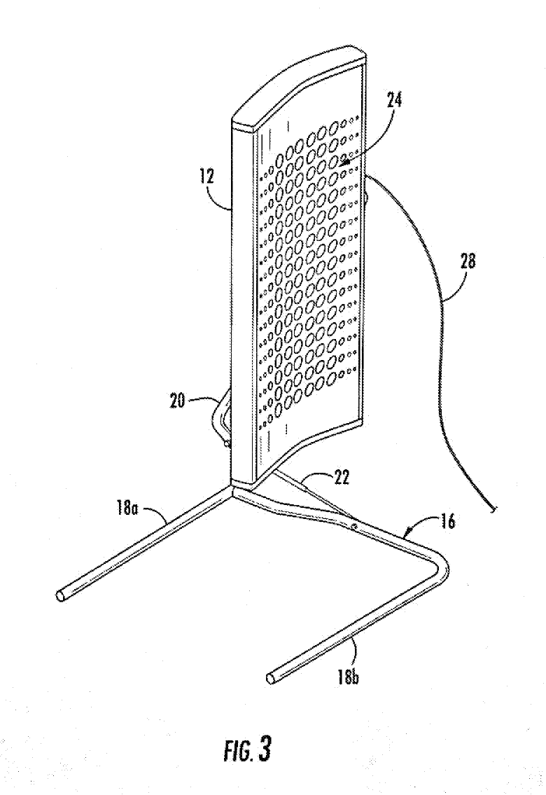

Because the canopy 12 is movable and pivotable relative of the stand 14, it may be positioned close to the patient (e.g. within 2″ of the patient's body) for an LLLT treatment described further below. Also, because the canopy 12 may be oriented into a vertical position as shown in FIG. 3, a person or athlete may be treated while in a standing position rather than lying down on a physical therapy bed if a patient is unable to lie down due to an injury or paralysis. The canopy 12 may be rotated vertically in order to treat a patient that is unable to lie down.

The canopy 12 includes a number of emitters 24 configured and arranged to emit light in wavelengths of between about 600 nm and about 1200 nm. The emitters 24 number in the hundreds. In one embodiment 361 emitters are used. In another embodiment between about 2000 and about 4000 emitters 24 are used. The number of emitters 24 may be varied depending upon the total power output desired. For instance power density may range from about 25 mW/cm² to about 150 mW/cm² and have a fluence range from about 50 J/cm² to about 250 J/cm². The emitters 24 may be configured to emit a constant amount of light or to pulse the light. If pulsed, the frequency of the pulsed light may be settable between about 2.5 Hz to about 25 KHz.

The emitters 24 may be light emitting diodes, lasers, and the like provided they can emit light within one or more of the parameters described above. The canopy 12 further include heat management unit 26 that has a number of fans and ducts configured and arranged to purge heat generated by the emitters 24. Power to the emitters 24 and heat management unit 26 is supplied through a power chord 28.

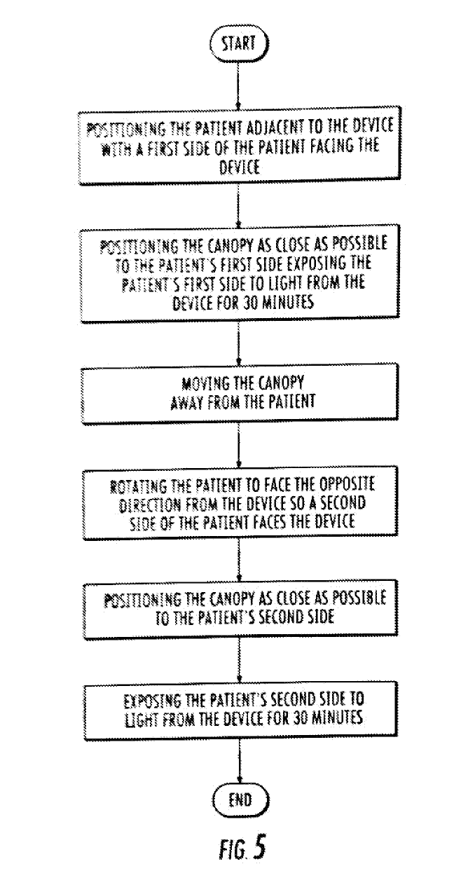

Referring to FIG. 5, during treatment with the device 10, in a first step, a full evaluation of a patient and their condition is performed, the patient then fully undresses and is placed supine on the bed. The patient is positioned adjacent to the device 10 either on a physical therapy bed or standing. One side of the patient, typically the front or the back, is treated first and is referred to as the first side. The opposite side of the patient is referred to as the second side.

The canopy 12 is then positioned as close as possible to the patient, e.g. within 2″ of the patient's body and the patient is given protective eyewear before the emitters 24 are turned on. The first side is treated by exposure of the first side to light from the emitters 24 for ten to thirty minutes.

At the end of the timed light therapy from the emitters 24, the emitters 24 are turned off and the canopy 12 is moved away from the patient. The patient is then re-evaluated. The patient is then rotated to the prone position. The canopy 12 is then positioned as close as possible to the patient, e.g. within 2″ of the patient's body and the patient is again given protective eyewear before the emitters 24 are turned on. The second side is treated by exposure of the second side to light from the emitters 24 for a time ranging from about ten to thirty minutes.

After the end of the timed light therapy, the emitters 24 are turned off and the treatment is complete. The patient is then re-evaluated for muscle recovery therapy and use of hand held low level laser system. The muscle recovery therapy involves evaluation of the patient for inflammation, pain, nerve issues and muscle condition. The patient may repeat the treatment three times a week for up to three consecutive months. For other indications, treatments form four to eight weeks may be prescribed. The duration of exposure may range from about ten to thirty minutes per side of the patient.

The advantage of the entire body being treated at once is that treatment times are significantly diminished while efficacy of the treatment is enhanced. This provides incredible benefit to certain populations of people—stroke patients, athletes, patients with fibromyalgia, chronic fatigue syndrome, patients with neurological disorders where a large part of the body is affected (Guillan-Barre syndrome, multiple sclerosis, Parkinson's dx).

Therefore, it can be seen that the present invention provides a unique solution to the problem of providing a device and method for applying a whole body LLLT treatment.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A device for application of photobiomodulation treatment, comprising
a canopy having a plurality of emitters configured and arranged to emit light in wavelengths between about 600 nm and about 1200 nm,
a stand supporting the canopy, and
the canopy configured and arranged pivot and raise and lower relative to the stand.

2. The device of claim 1, wherein the plurality of emitters is 361.

3. The device of claim 1, wherein the plurality of emitters is configured and arranged to be about 2000 to about 4000 emitters.

4. The device of claim 1, wherein the emitters are light emitting diodes.

5. The device of claim 1, wherein the emitters are configured and arranged to pulse between about 2.5 Hz to about 25 KHz.

6. The device of claim 1, wherein the emitters are configured and arranged to have a power output between about 25 mW/cm² to about 150 mW/cm².

7. The device of claim 1, wherein the emitters are configured and arranged to have fluence range from about 50 J/cm² to about 250 J/cm².

8. The device of claim 1, wherein the stand further includes a base, an arm pivotably attached to the base at a first end and the canopy at a second end.

9. The device of claim 8, further comprising a hydraulic cylinder connected to the arm and the base configured and arranged to support to support the arm in a desired position.

10. A method of treating a patient, comprising
exposing an entire first side of a patient to emitted light between about 600 nm and 1200 nm for a therapeutically effective duration, and
exposing an entire second side of a patient to emitted light between about 600 nm and 1200 nm for a therapeutically effective duration.

11. The method of claim 10, wherein the therapeutically effective duration is from ten to thirty minutes.

12. The method of claim 10, wherein the therapeutically effective duration is between ten and thirty minutes total.

13. The method of claim 10, wherein the emitted light is pulsed between about 2.5 Hz to about 25 KHz.

14. The method of claim 10, further comprising emitting the emitted light at a power output between about 25 mW/cm² to about 150 mW/cm².

15. The method of claim 10, further comprising emitting the emitted light at a fluence range from about 50 J/cm² to about 250 J/cm².

16. The method of claim 10, further comprising repeating the steps of exposing the first side and exposing the second side three times a week for about three months.

17. The method of claim 10, further comprising repeating the steps of exposing the first side and exposing the second side three times a week for between about four weeks and about eight weeks.
